(19) United States
(12) Patent Application Publication
(10) Pub. No.: US 2025/0074977 A1
(43) Pub. Date: Mar. 6, 2025

(54) METHOD FOR IMPROVING WOUND HEALING

(71) Applicant: NATIONAL YANG MING CHIAO TUNG UNIVERSITY, Taipei (TW)

(72) Inventors: Ting-Ting CHANG, Taipei (TW); Jaw-Wen CHEN, Taipei (TW)

(73) Assignee: NATIONAL YANG MING CHIAO TUNG UNIVERSITY, Taipei (TW)

(21) Appl. No.: 18/459,866

(22) Filed: Sep. 1, 2023

(51) Int. Cl. C07K 16/24; A61K 31/713; A61P 17/02; C12N 15/113

(52) U.S. Cl. CPC C07K 16/24; A61K 31/713; A61P 17/02; C12N 15/113; A61K 2039/505

(57) ABSTRACT

Provided is a method for improving would healing, including administering an effective amount of a chemokine C-C motif ligand 7 (CCL7) antagonist to a subject in need thereof to inhibit CCL7 activity.

### BACKGROUND OF THE INVENTION

### 1. Technical Field

The present disclosure relates to a method for improving wound healing, and particularly to a method for the prevention and/or treatment of chronic wounds.

### 2. Description of Related Art(s)

Wounds generally heal over time, but wounds in diabetic patients do not heal quickly and chronic wounds are common in diabetic patients. This has a negative impact on both the patient and the healthcare system. Considering the increasing prevalence of diabetes, it will be a significant medical, social, and economic burden in the near future. Therefore, there is a need to provide alternatives to the currently available treatments, which, although diverse, do not guarantee a rapid and definitive repair process.

In diabetic patients, vasculopathy and poor wound vascularization can lead to prolonged wound healing and amputation. In addition to risk factors such as hypertension, lipids, and glucose, diabetic vasculopathy is associated with a systemic vascular inflammatory response. Poor wound healing among diabetic patients accounts for the majority of patients with chronic wounds. The main reason for this is low wound angiogenesis, so nutrients do not work at the wound site; therefore, how to promote wound angiogenesis is an important issue. However, there are very few U.S. FDA-approved drugs on the market to improve chronic wounds, especially those recommended for patients with diabetic vascular disease and infection, such as diabetic foot patients; therefore, some innovative drugs still need further development.

The majority of the diabetic population is expected to suffer from foot ulcers during their lifetime. These ulcers tend to be chronic in nature as they do not heal or heal extremely slowly. Diabetic foot ulcers are a serious problem for diabetic patients, as most diabetic foot ulcers would eventually require amputation due to peripheral vascular lesions.

### SUMMARY

In view of the foregoing, the present disclosure provides a method for improving would healing, comprising administering an effective amount of a chemokine C-C motif ligand 7 (CCL7) antagonist to a subject in need thereof to inhibit CCL7 activity.

In one embodiment of the present disclosure, the CCL7 antagonist is selected from a group consisting of a CCL7 neutralizing antibody, a CCL7 RNA interference (RNAi) agent, a C-C chemokine receptor type 1 antagonist, a C-C chemokine receptor type 2 antagonist, a C-C chemokine receptor type 3 antagonist, a C-C chemokine receptor type 5 antagonist, and a combination thereof.

In one embodiment of the present disclosure, the wound is a chronic wound.

In one embodiment of the present disclosure, the subject suffers from diabetic foot ulcer.

In one embodiment of the present disclosure, the effective amount of the CCL7 antagonist is from about 0.01 μg/kg to about 100 mg/kg. In another embodiment of the present disclosure, the effective amount of the CCL7 antagonist is from about 0.1 μg/kg to about 1 mg/kg.

In one embodiment of the present disclosure, the CCL7 antagonist is administered to the subject orally, sublingually, parenterally, rectally, intraperitoneally, intravenously, intradermally, intrapulmonarily, intramuscularly, subcutaneously, intrapleurally, topically, intranasally, or transdermally.

In one embodiment of the present disclosure, the administering enhances angiogenesis in the subject.

In one embodiment of the present disclosure, the administering protects endothelial cell functions in the subject.

In one embodiment of the present disclosure, a tube formation ability is improved.

In one embodiment of the present disclosure, a migration ability of endothelial cells is improved.

In one embodiment of the present disclosure, an expression of at least one inflammatory factor selected from the group consisting of G-CSF, IL-1B, IL-la, IL-2, IL-6, IL-8, IL-11, IL-17, IL-18, IFN-α, IFN-β, IFN-γ, TNF-α, TNF-β, and a combination thereof is decreased, preferably, the inflammatory factor is selected from the group consisting of IL-1B, IL-6, TNF-α, and a combination thereof.

In one embodiment of the present disclosure, an expression of an angiogenic factor is increased. In another embodiment of the present disclosure, the angiogenic factor comprises VEGF, SDF-1, EGF, Ang, EPO, FGF, GDF, or a combination thereof, preferably the angiogenic factor is at least one selected from the group consisting of VEGF, SDF-1, EGF, Ang, EPO, FGF, GDF, and a combination thereof, more preferably, the angiogenic factor is VEGF, SDF-1, or a combination thereof. In yet embodiment of the present disclosure, VEGF can be any one of VEGF family, such as VEGF-A, VEGF-C, VEGF-D, or placental growth factor (PIGF).

In the present disclosure, by using the CCL7 antagonist, the method provided in the present disclosure may improve the functions of human dermal microvascular endothelial cells (HDMECs), thereby promoting wound healing. The method of using a CCL7 antagonist of the present discourse is useful in improving the tube formation ability and/or the migration ability of endothelial cells, and thus is effective in improving would healing.

Further, the present disclosure also provides a use of an effective amount of the aforementioned CCL7 antagonist in the manufacture of a medicament for improving would healing in a subject in need thereof, and the CCL7 antagonist inhibits CCL7 activity. Also provided is an effective amount of the aforementioned CCL7 antagonist for use in improving would healing in a subject in need thereof, and the CCL7 antagonist inhibits CCL7 activity.

Other objectives, advantages and novel features of the invention will become more apparent from the following detailed description when taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following description of the embodiments, with reference made to the accompanying drawings.

FIGS. 1A and 1B illustrate the upregulation of CCL7 in high glucose-stimulated HDMECs.

FIGS. 2A-2D show that the expression of inflammatory factors (IL-1B, IL-6, and TNF-α) is enhanced in high glucose-stimulated HDMECs.

FIGS. 3A-3C show that the expression of angiogenic factors (VEGF and SDF-1) is decreased in high glucose-stimulated HDMECs.

FIGS. 4A-4D show that the expression of inflammatory factors (TNF-α, IL-1B and IL-6) is enhanced by direct stimulation of CCL7.

FIGS. 5A-5C show that the expression of angiogenic factors (VEGF, and SDF-1) is decreased by direct stimulation of CCL7.

FIGS. 6A and 6B illustrate that the tube formation ability of HDMECs is impaired by the administration of CCL7.

FIGS. 7A and 7B illustrate that the migration ability of HDMECs is impaired by the administration of CCL7.

FIGS. 8A and 8B show that high glucose induced CCL7 is knocked down by the administration of CCL7 siRNA (siCCL7).

FIGS. 9A and 9B show that the expression of inflammatory factors (IL-1B, IL-6, and TNF-α) is decreased by the administration of siCCL7.

FIGS. 10A and 10B show that the expression of angiogenic factors (VEGF, and SDF-1) is increased by the administration of siCCL7.

FIGS. 11A and 11B illustrate that the tube formation ability of HDMECs is improved by the administration of siCCL7.

FIGS. 12A and 12B illustrate that the migration ability of HDMECs is improved by the administration of siCCL7.

FIGS. 13A-13D illustrate the inhibition of CCL7 by neutralizing antibody improves wound repair in db/db type 2 diabetic mice. FIG. 13A shows the wound areas.

FIG. 13B is a quantitative result showing closure rates of the wounds. FIG. 13C is an image of H&E staining showing the improvement of wound healing in wound sections of the CCL7 antibody-treated diabetic mice. FIG. 13D is an image of CD31 immunostaining indicating that the detection of higher CD31 expressions in the wound area of the CCL7 antibody-treated diabetic mice than the untreated diabetes mellitus (DM) mice.

### DETAILED DESCRIPTION OF THE PREFERRED EMBODIMENTS

The following examples are used to illustrate the present disclosure. A person skilled in the art can easily conceive of the other advantages and effects of the present disclosure based on the invention of the specification. The present disclosure can also be implemented or applied as described in various examples. It is possible to modify or alter the following examples for carrying out the present disclosure without violating its spirit and scope, for different aspects and applications.

It is further noted that the singular forms “a,” “an,” and “the” as used in the present disclosure include plural referents unless expressly and unequivocally limited to one referent. The term “or” is used interchangeably with the term “and/or” unless the context clearly indicates otherwise.

As used herein, the term “comprising” or “comprises” is used in reference to compositions, methods, and respective component(s) thereof, which are essential to the present disclosure, yet open to the inclusion of unspecified elements, whether essential or not.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Some terms may be arbitrarily selected in this disclosure, and in this case, the meaning of the selected terms will be described in detail in the descriptions of the present disclosure. The terms used herein have to be defined based on the meaning of the terms together with the descriptions throughout the specification.

The present disclosure provides a method for improving would healing, comprising administering an effective amount of a chemokine C-C motif ligand 7 (CCL7) antagonist to a subject in need thereof to inhibit CCL7 activity.

In one embodiment of the present disclosure, the CCL7 antagonist, also known as monocyte-specific chemokine (MCP)-3 antagonist, is capable of preventing the binding of CCL7 to its receptor. In another embodiment, the CCL7 antagonist is an agent that inhibits intracellular signaling generated by the binding of CCL7 to its receptor. For example, the CCL7 antagonist may be directed against at least one of CCL7 and the receptor of CCL7, thereby blocking the CCL7 signaling. As used herein, the receptor of CCL7 includes, but is not limited to, CCR1, CCR2, CCR3, CCR5. As used herein, the terms “CCR1” or “CCR1 receptor”, “CCR2” or “CCR2 receptor”, “CCR3” or “CCR3 receptor”, and “CCR5” or “CCR5 receptor” are used interchangeably and have their general meaning in the art. The CCR1, CCR2, and CCR3 receptors may be from any source but typically a mammalian (e.g., human or non-human primate) CCR1, CCR2, CCR3, and CCR5 receptor. In some embodiments of the present disclosure, the CCR1, CCR2, CCR3 and CCR5 receptors are human receptors.

As used herein, the term “CCL7” has its general meaning in the art. CCL7 is a natural ligand of the CCR3 receptor and may be from any source, but typically is a mammalian (e.g., human or non-human primate) CCL7. In one embodiment of the present disclosure, the CCL7 is a human CCL7.

As used herein, the term “CCL7 antagonist” includes any entity that, upon administration to a subject, results in inhibition or down-regulation of a biological activity associated with CCL7 in the subject, including any of the downstream biological effects otherwise resulting from the binding of CCL7 to its receptor. The 5 CCL7 antagonist includes any agent that may inhibit CCL7 activity or block activation of the receptor of CCL7 or any of the downstream biological effects of activation of the receptor of CCL7. Such a CCL7 antagonist includes any agent that is able to interact with CCL7 so that its normal biological activity is prevented or reduced. For example, said agent may be a small organic molecule or an antibody directed against CCL7, such as a CCL7 neutralizing antibody, which can block the interaction between CCL7 and its receptor, or which can block the activity of CCL7. The CCL7 antagonist may also be a small molecule or an antibody directed against the receptor of CCL7, which may act by occupying the ligand binding site or a portion thereof of the receptor, thereby making the receptor inaccessible to its ligand, CCL7.

The phrase “an effective amount” refers to the amount of an active ingredient that is required to result in a reduction, inhibition, or prevention of the disorder, abnormality, or symptom in the individual. An effective amount will vary, as recognized by those skilled in the art, depending on routes of administration, excipient usage, and the possibility of co-usage with other therapeutic treatments.

The term “subject” as used herein is interchangeable with “individual” and includes a single biological organism, including, but not limited to, animals such as vertebrates, mammals, or human beings. The term “a subject in need thereof” refers to a subject expressing or suffering from one or more symptoms related to chronic wounds. An appropriately qualified person or physician is able to identify such an individual in need of treatment using standard testing protocols or guidelines. The same testing protocols or guidelines may also be used to determine whether there is an improvement to the individual's disorders or symptoms or determine the most effective dose of improving chronic wounds to be administered to an individual in need of the treatment.

The term “improvement” as used herein refers to prevention or reduction in the severity or frequency, to whatever extent, of one or more of the symptoms or abnormalities expressed by the individual diagnosed with chronic wounds. The improvement is either observed by the individual taking the treatment themselves or by another person.

The methods and the CCL7 antagonist of the present disclosure may be used to treat a variety of conditions that would benefit from improvement of the tube formation ability of endothelial cells, the improvement of the migration ability of endothelial cells, decreased expression of inflammatory factors, and/or increased expression of angiogeni factors.

The term “angiogenesis” indicates the growth or formation of blood vessels. Angiogenesis includes the growth of new blood vessels from pre-existing vessels, as well as vasculogenesis, which refers to spontaneous blood-vessel formation, and intussusception, which refers to new blood vessel formation by splitting off existing ones. Angiogenesis encompasses “neovascularization,” “regeneration of blood vessels,” “generation of new blood vessels,” and “revascularization.”

As used herein, the term “treating” or “treatment” refers to obtaining a desired pharmacologic and/or physiologic effect, e.g., improving wound healing, or stimulation of angiogenesis. The effect may be prophylactic in terms of completely or partially preventing a disease or symptom thereof or may be therapeutic in terms of completely or partially curing, alleviating, relieving, remedying, ameliorating a disease, or an adverse effect attributable to the disease.

As used herein, the term “wound” includes surgical incisions and wounds caused by accidental trauma or pathologies. The term also includes venous stasis ulcer, burns, delayed wound healing observed during corticoid treatments, delayed wound healing observed in elderly (aging defect), stress, delayed wound healing observed in diabetic patients, and epithelialization defects of surgical scars or following skin grafts. In particular, the term “wound” as used herein refers to any wound resulting from diabetes, such as diabetic foot ulcer.

As used herein, the phrase “an effective amount” refers to the amount of an active agent (e.g., CCL7 antagonist) that is required to confer a desired therapeutic effect (e.g., a desired level of angiogenic stimulation) on the treated subject. Effective doses will vary, as recognized by those skilled in the art, depending on routes of administration, excipient usage, the possibility of co-usage with other therapeutic treatment, and the condition to be treated.

In one embodiment of the present disclosure, the effective amount of the CCL7 antagonist is from about 0.01 μg/kg to about 100 mg/kg, such as from about 0.05 μg/kg to about 90 mg/kg, from about 0.1 μg/kg to about 80 mg/kg, from about 0.2 μg/kg to about 70 mg/kg, from about 0.4 μg/kg to about 60 mg/kg, from about 0.6 μg/kg to about 50 mg/kg, from about 0.7 μg/kg to about 40 mg/kg, from about 0.8 μg/kg to about 30 mg/kg, from about 0.9 μg/kg to about 20 mg/kg from about 1 μg/kg to about 10 mg/kg, from about 1.5 μg/kg to about 5 mg/kg, from about 2 μg/kg to about 1 mg/kg, from about 2.5 μg/kg to about 500 μg/kg, from about 3 μg/kg to about 400 μg/kg, from about 3.5 μg/kg to about 300 μg/kg, from about 4 μg/kg to about 200 μg/kg, from about 4.5 μg/kg to about 100 μg/kg, from about 5 μg/kg to about 50 μg/kg, from about 5.5 μg/kg to about 40 μg/kg, from about 6 μg/kg to about 30 μg/kg, from about 6.5 μg/kg to about 20 μg/kg, from about 7 μg/kg to about 10 μg/kg, from about 7.5 μg/kg to about 9.5 μg/kg, or from about 8 μg/kg to about 9 μg/kg. In another embodiment, the effective amount of the CCL7 antagonist has a lower limit chosen from 0.01 μg/kg, 0.05 μg/kg, 0.1 μg/kg, 0.5 μg/kg, 1 μg/kg, 2 μg/kg, 3 μg/kg, 4 μg/kg, and 5 μg/kg, and an upper limit was chosen from 100 mg/kg, 90 mg/kg, 80 mg/kg, 70 mg/kg, 60 mg/kg, 50 mg/kg, 40 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 10 μg/kg, 9 μg/kg, 8 μg/kg, 7 μg/kg, and 6 μg/kg.

In one embodiment of the present disclosure, the CCL7 antagonist is administered 1 to 2 times over a period of 2 to 4 days. In another embodiment, the CCL7 antagonist is administered 8 to 15 times over a period of 3 to 5 weeks. For example, the CCL7 antagonist is administered 3 times over a week, or 10 times over a period of 4 weeks. In yet another embodiment, the CCL7 antagonist is administered 1-4 weeks apart, such as one week, 8 days, 9 days, 10 days, 11 days, 12 days, 13 days, two weeks, 15 days, 16 days, 17 days, 18 days, 19 days, 20 days, three weeks, 22 days, 23 days, 24 days, 25 days, 26 days, 27 days, or four weeks.

As used herein, the term “administering” or “administration” refers to the placement of an active agent (e.g., CCL7 antagonist) into a subject by a method or route which results in at least partial localization of the active agent at a desired site such that a desired effect is produced. The active agent described herein can be administered by any appropriate route known in the art including, but not limited to, oral or parenteral routes, including oral, sublingual, parenteral, rectal, intraperitoneal, intravenous, intradermal, intrapulmonari, intramuscular, subcutaneous, intrapleural, topical, intranasal, or transdermal routes.

In one embodiment of the present disclosure, the CCL7 antagonist may be presented in a pharmaceutical composition to be administered to the subject. In certain embodiments, the present disclosure provides a pharmaceutical composition for stimulating angiogenesis, comprising the CCL7 antagonist and a pharmaceutically acceptable carrier. The pharmaceutical composition provided in the present disclosure may efficiently prevent or treat wound healing.

In one embodiment of the present disclosure, the pharmaceutically acceptable carrier may be a diluent, a disintegrant, a binder, a lubricant, a glidant, a surfactant, or a combination thereof.

In one embodiment of the present disclosure, the pharmaceutical composition is a sterile injectable composition, which may be a solution or suspension in a non-toxic parenterally acceptable diluent or solvent. Among the acceptable vehicles and solvents that can be employed are 1,3-butanediol, mannitol, water, Ringer's solution, and isotonic sodium chloride solution. In addition, fixed oils are conventionally employed as a solvent or suspending medium (e.g., synthetic mono- or di-glycerides). Fatty acids, such as oleic acid and its glyceride derivatives, are useful in the preparation of injectables, as naturally pharmaceutically acceptable oils, such as olive oil and castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens and Spans or other similar emulsifying agents or bioavailability enhancers which are commonly used in the manufacture of pharmaceutically acceptable solid, liquid, or other dosage forms can also be used for the purpose of formulation.

The carrier in the pharmaceutical composition must be “acceptable” in the sense that it is compatible with the active agent of the composition (and can be capable of stabilizing the active agent) and not deleterious to the subject to be treated. One or more solubilizing agents can be utilized as pharmaceutical excipients for delivery of an active compound. Examples of other carriers include colloidal silicon oxide, magnesium stearate, cellulose, and sodium lauryl sulfate.

Many examples have been used to illustrate the present disclosure. The examples below should not be taken as a limit to the scope of the present disclosure.

### Materials and Methods

### In Vitro Study

### Cell Culture

Human dermal microvascular endothelial cells (HDMECs, ScienCell, Catalog #2000, Carlsbad, CA, USA) were cultured with Endothelium Cell Medium (ECM) containing VEGF and 1% penicillin/streptomycin (Sigma-Aldrich, P4333, Darmstadt, Germany), and the cultured dishes were coated with fibronectin before being used.

Some cells were directly treated with recombinant human CCL7 protein (R&D Systems, 282-P3, Minneapolis, MN, USA) in 0.1 or 1 ng/ml for 48 hours.

Some cells were treated with siRNA Control (Santa Cruz Biotechnology, sc-37007, Dallas, TX, USA) and CCL7 siRNA (Santa Cruz Biotechnology, sc-72035, Dallas, TX, USA) to evaluate the direct endogenous role of CCL7. To mimic the hyperglycemia in DM, high glucose (HG; Sigma-Aldrich, St. Louis, MO, USA) in a concentration of 25 mM was administered to HDMECs.

### Western Blot

Samples were extracted using lysis buffer, and proteins were separated in 8-12% (v/v) SDS-PAGE gels. After electrophoresis (Bio-Rad Laboratories, Hercules, CA, USA), the proteins were transferred onto nitrocellulose membranes (Millipore, Darmstadt, Germany). The membranes were incubated with primary antibodies against CCL7 (R&D Systems, MAB282; Minneapolis, MN, USA), VEGF (Santa Cruz Biotechnology, sc-152; Dallas, TX, USA), stromal cell-derived factor-1 (SDF-1; Cell Signaling, 3740S; Boston, MA, USA), TNF-α (Cell Signaling, 3707S; Boston, MA, USA), interleukin-6 (IL-6; Cell Signaling, 12153S; Boston, MA, USA), interleukin-1ß (IL-1B; Santa Cruz Biotechnology, sc-7884; Dallas, TX, USA), and β-actin (Merck, MAB1501, Darmstadt, Germany) at 4° C. overnight. After washing three times, the membranes were incubated with HRP-conjugated secondary antibodies (1:1000) for 1 hour at room temperature. Finally, the membranes were visualized by using the ECL kit.

### Migration Assay

The Transwell (Corning, Tewksbury, MA, USA) migration assay was used to analyze the migrating ability of HDMECs after treatments. The cells (1×10⁴ cells) were suspended in a serum-free cultured medium. HDMECs were incubated with a medium containing 25 mM glucose for 2 days after treatment. The cells were seeded on the upper chamber of a 24-well Transwell plate with a polycarbonate membrane, and the cells migrated toward the lower chamber containing 600 μL cultured medium with FBS at 37° C. and 5% CO2. After 18 hours, the migrated cells were fixed in 4% paraformaldehyde and stained with hematoxylin solution. The images were captured by using high-power (×100) microscope.

### Tube Formation Assay

HDMECs were seeded into a 6-well plate at each well until a monolayer was formed, and the combined treatment was then conducted. Cells were collected by trypsinization, and 1×10+ cells/well were seeded into ECMatrix gel (Merck, ECM625, Darmstadt, Germany) in 96-well plates, in 100 μL cultured medium with 10% FBS, for 16 hours at 37° C. and 5% CO2. The images were captured using a high-power (×40) microscope. The numbers of the formed tubes of cells were calculated by using Image-Pro Plus (Media Cybernetics, Inc. Rockville, MD, USA).

### In Vivo Study

### Animal Models of DM

Six-week-old male BKS.Cg-m+/+Leprdb/JNarl mice were purchased from the National Laboratory Animal Center (Taipei, Taiwan). Mice were all acclimated for 2 weeks before experiments. For the treatment of wound healing, mice were injected intraperitoneally with CCL7 neutralizing antibody (0.1 or 1 μg; R&D, AF-456, Minneapolis, MN, USA) or IgG antibody (1 μg; R&D, AB-108, Minneapolis, MN, USA) three times per week. Animals were raised according to the regulations of the Animal Care Committee of National Yang Ming Chiao Tung University (Taipei, Taiwan). The animal study was approved by the Animal Care Committee of National Yang Ming Chiao Tung University (IACUC No. 1100419).

### Wound Healing Assay

A wound healing assay is performed to explore the role of CCL7 in cutaneous wound recovery in DM mice. Mice were anesthetized via inhaled isoflurane. The back skin was shaved and cleaned with antibacterial soap solution and 75% alcohol. Circular full-thickness excisional wounds of 3 mm diameter were generated by biopsy punch without muscle injury. Wounds were recorded using a digital camera (Nikon, Tokyo, Japan) after being generated.

### Histological and Immunohistochemistry Analysis

The wound samples were fixed with 4% paraformaldehyde for 24 hours, dehydrated in graded alcohols, and then embedded in paraffin wax. The tissues were sectioned into samples of 5 μm thickness. Sections were dried overnight and stained with hematoxylin and eosin stain for histological analysis. In addition, histological analyses of capillary densities were determined. Antigen retrieval was performed using 0.05 M sodium citrate buffer. Slides were then incubated at 4° C. overnight with the primary antibody to detect CD31 (Abcam, 124432, Waltham, MA, USA). The sample was washed with PBS solution and incubated with a secondary antibody (rabbit) for 2 hours at room temperature. The CD31-positive sites were shown in dark brown. Three cross-sections were analyzed for each animal; ten different fields from each tissue preparation were randomly selected, and visible capillaries were counted.

### Statistical Analysis

Results are expressed as the mean±standard deviation (SD). Data sets were analyzed with the unpaired Student's t test, followed by a Scheffe's multiple-comparison post hoc test. Statistical significance was set at p-values<0.05.

### Results

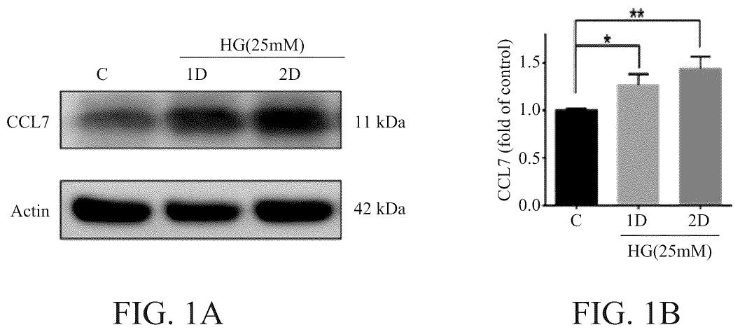

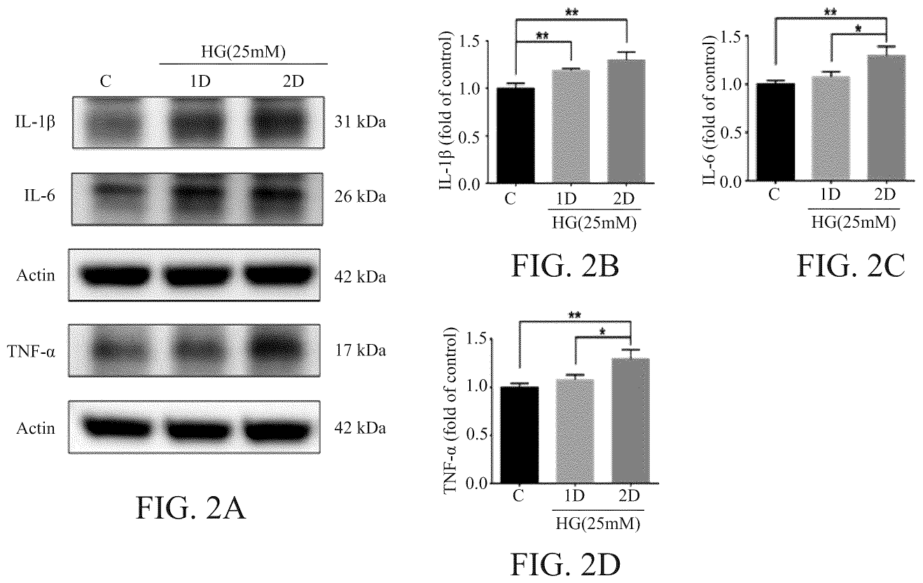

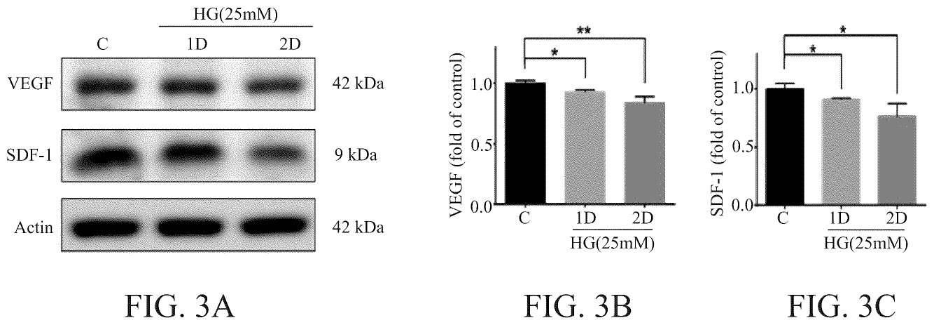

HDMECs isolated from adult skins were used to explore the direct effects of CCL7 in the wound healing process under the pathological condition in vitro. FIGS. 1-3 show the results of Western blotting and statistical analyses of CCL7 (FIGS. 1A and 1B; n=3), inflammatory factors of IL-1β, IL-6, TNF-α (FIGS. 2A-2D; n=3), along with angiogenic factors of VEGF, and SDF-1 (FIGS. 3A-3C; n=3) in HG-stimulated HDMECs. C represents untreated HDMECs. HG represents high glucose; 1D represents treatment for 1 day; 2D represents treatment for 2 days. * P<0.05, ** P<0.01.

As can be seen in FIGS. 1-3, CCL7 was upregulated in HG-stimulated HDMECs with enhanced inflammatory and decreased angiogenic factor expressions.

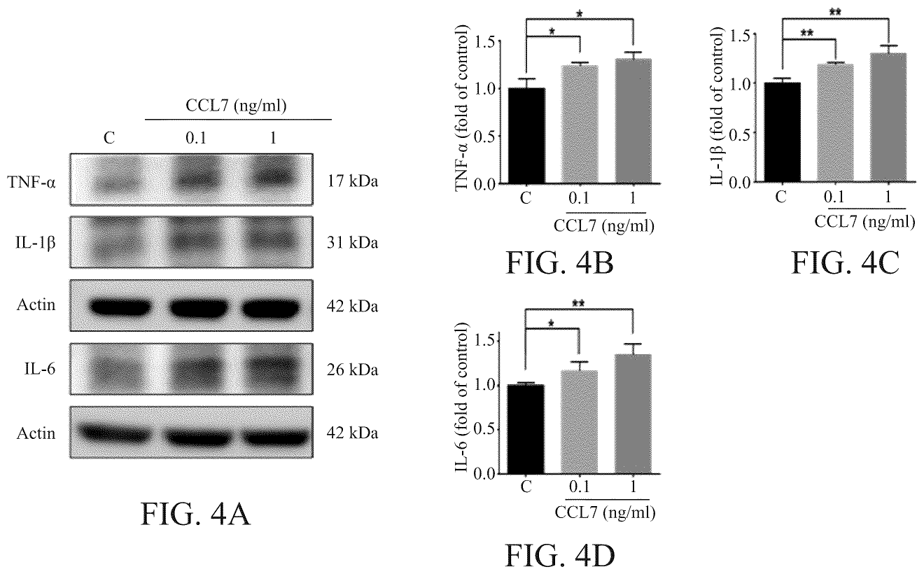

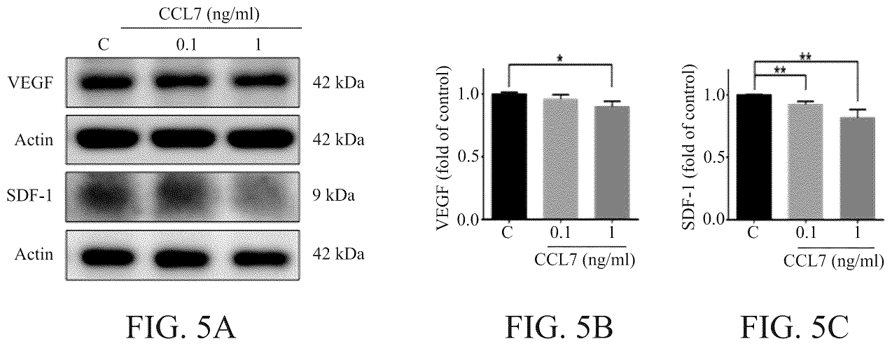

FIGS. 4 and 5 show the results of Western blotting and statistical analyses in connection with inflammatory factors of TNF-α, IL-1β, IL-6 (FIGS. 4A-4D; n=3), and angiogenic factors of VEGF, and SDF-1 (FIGS. 5A-5C; n=3). C represents untreated HDMECs. * P<0.05, ** P<0.01.

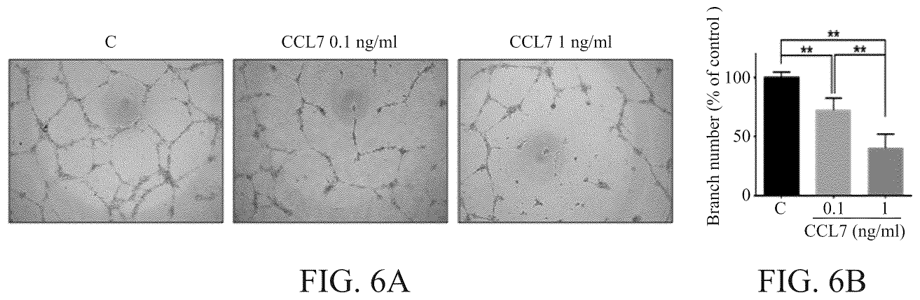

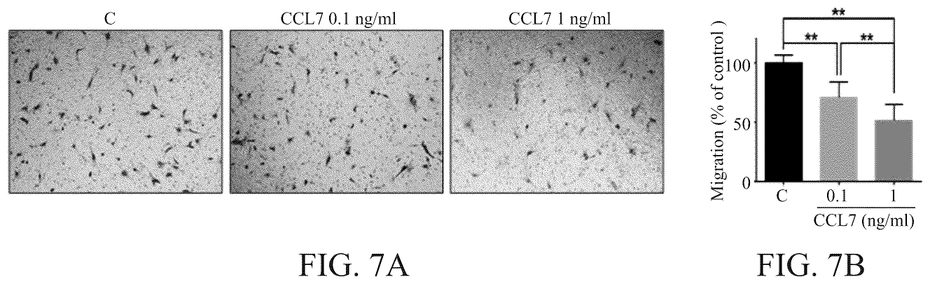

FIGS. 6 and 7 show the tube formation ability and the migration ability and quantitative analysis of HDMECs, respectively. The tube formation ability was impaired after the administration of CCL7 (FIGS. 6A and 6B; n=3). The migration ability was impaired after the administration of CCL7 (FIGS. 7A and 7B; n=3). C represents untreated HDMECs. * P<0.05, ** P<0.01.

From the results shown in FIGS. 4-7, direct stimulation of CCL7 would impair cell functions with enhanced expression of inflammatory factors and decreased expression of angiogenic factors in HDMECs, and the impaired cell functions including tube formation ability and migration ability in HDMECs.

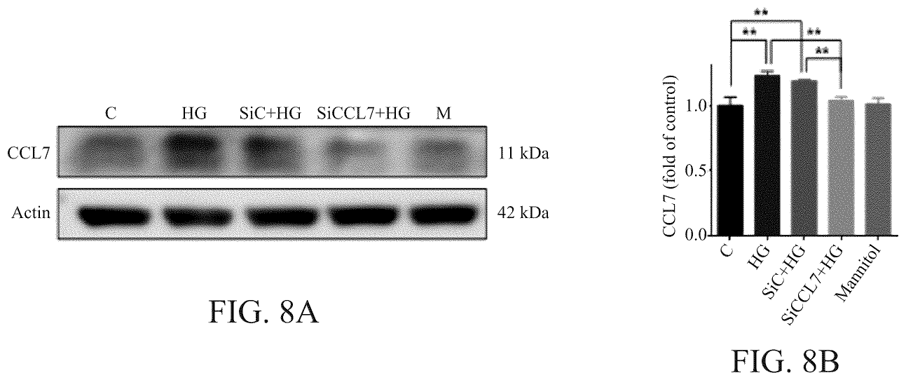

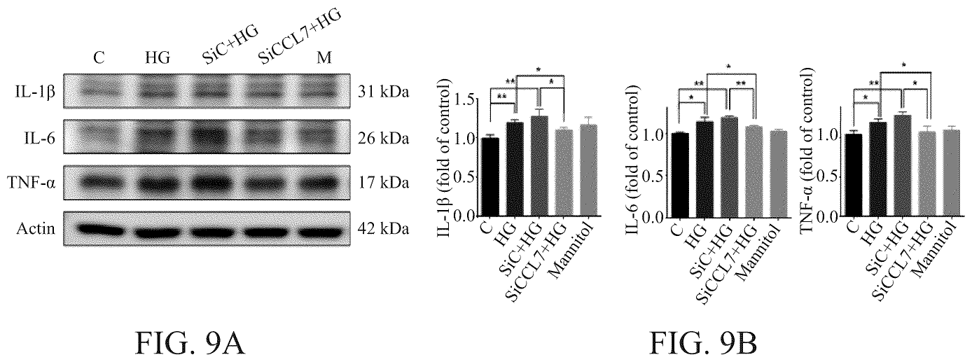

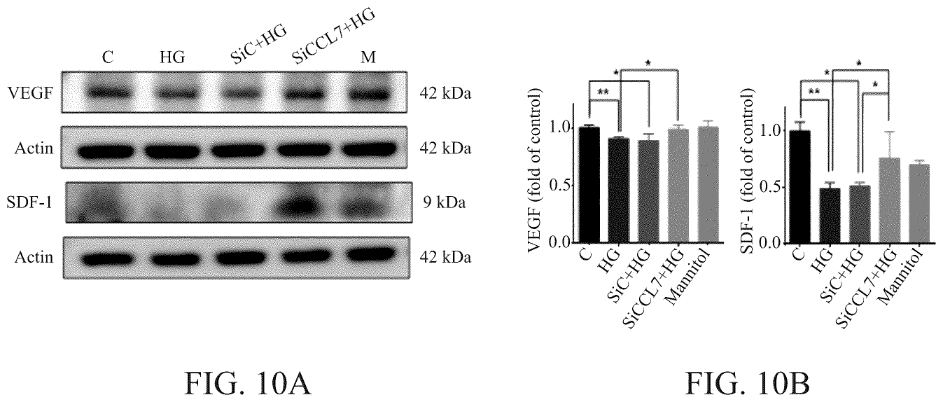

FIGS. 8-10 show the results of Western blotting and statistical analyses regarding CCL7 (FIGS. 8A and 8B and 1B; n=3), inflammatory factors of IL-1β, IL-6, TNF-α (FIGS. 9A and 9B; n=3), along with angiogenic factors of VEGF, and SDF-1 (FIGS. 10A and 10B; n=3) after the administration of siCCL7. C represents untreated HDMECs. HG represents high glucose treatment for 2 days. SiC+HG represents the treatment of siRNA Control (SiC) in combination with high glucose for 2 days. SiCCL7+HG represents the treatment of SiCCL7 in combination with high glucose for 2 days. M represents mannitol treatment for 2 days. * P<0.05, ** P<0.01.

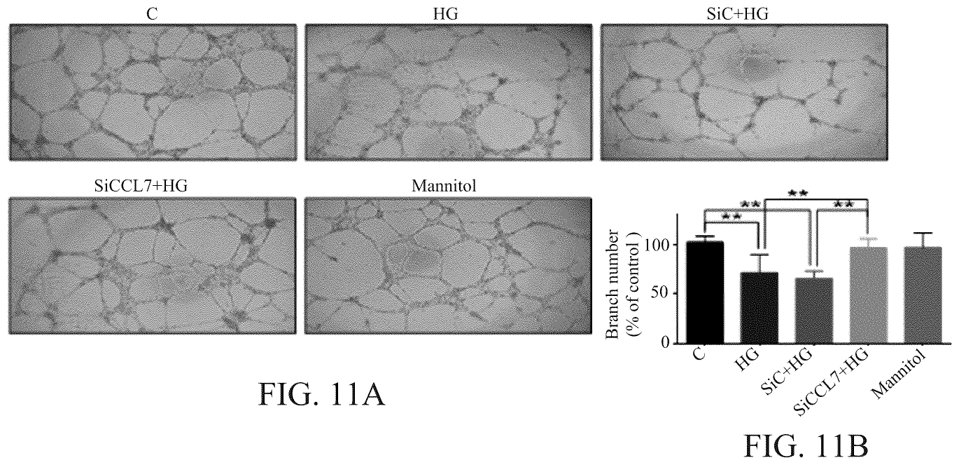

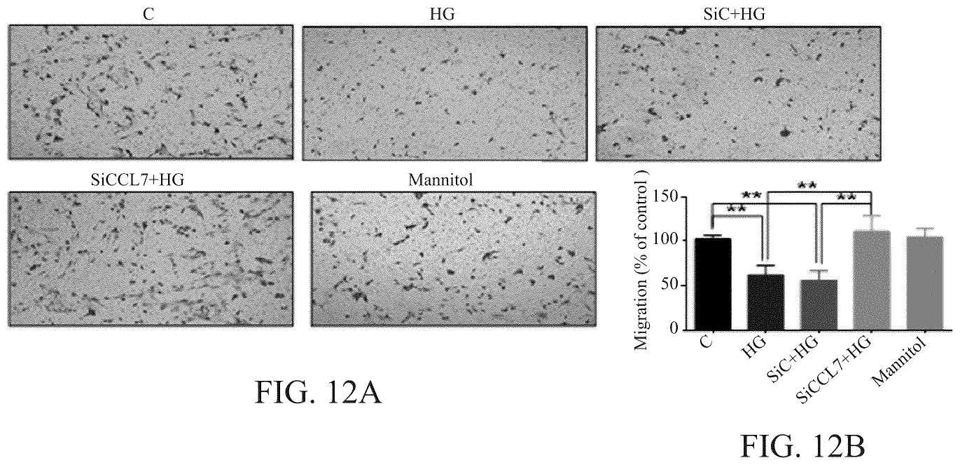

FIGS. 11 and 12 show the tube formation ability and the migration ability and quantitative analysis of HDMECs, respectively. The tube formation ability was improved after the administration of siCCL7 (FIGS. 11A and 11; n=3). The migration ability was also improved after the administration of siCCL7 (FIGS. 12A and 12B; n=3). C represents untreated HDMECs. HG represents high glucose treatment for 2 days. SiC+HG represents the treatment of siRNA Control (SiC) in combination with high glucose for 2 days. SiCCL7+HG represents the treatment of SiCCL7 in combination with high glucose for 2 days. M represents mannitol treatment for 2 days. * P<0.05, **P<0.01.

As can be seen in the results of FIGS. 8-12, the inhibition of CCL7 by siRNA would recover functions of HG-impaired HDMECs with increased angiogenic protein expressions.

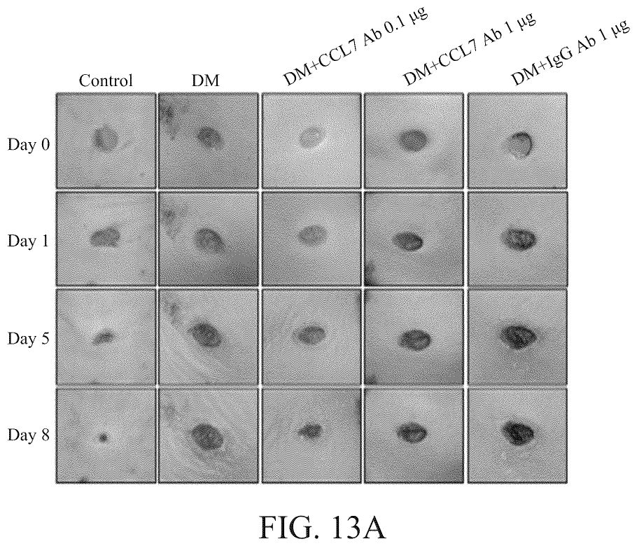

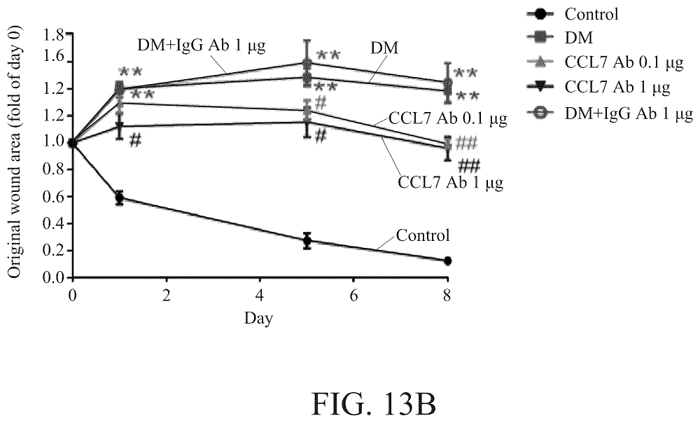

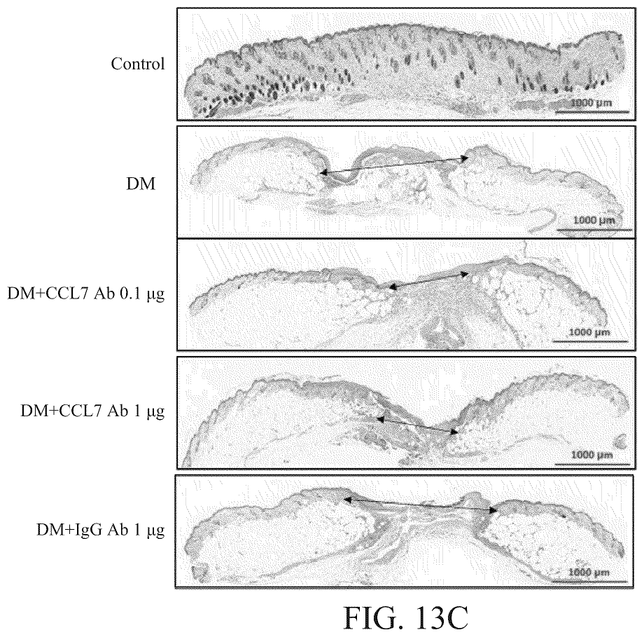

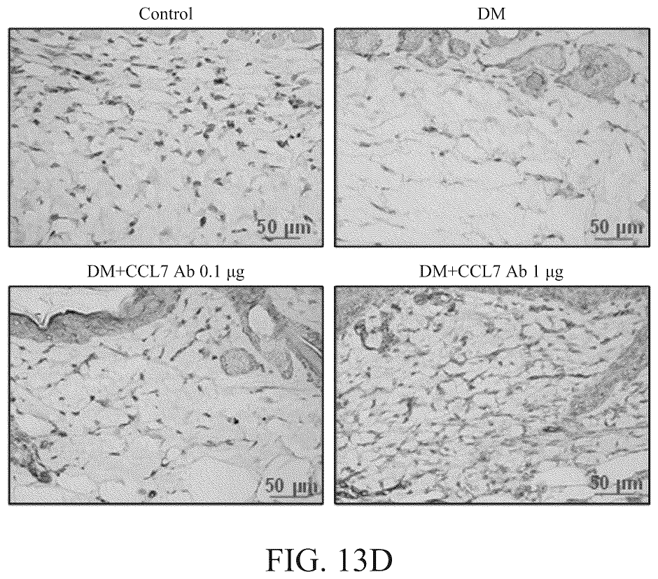

Please refer to FIGS. 13A and 13B. As the wound areas shown in FIG. 13A, the diabetic mice had a significant delayed wound repair post injury compared to the non-DM control group. The closure rates of 3-mm punch biopsies were measured (n=6), and the results were shown in FIG. 13B. * p<0.05, ** p<0.01 compared with control; #p<0.05, ##p<0.01 compared with DM. The accelerated rate of wound closure was observed in the CCL7 antibody-treated mice (i.e., DM+CCL7 Ab 0.1 μg and DM+CCL7 Ab 1 μg) compared with that in the untreated DM mice.

FIGS. 13C and 13D are representative images of H&E staining and CD31 immunostaining, respectively. As shown in the results of H&E staining, the improvement on the wound healing in the CCL7 antibody-treated diabetic mice can be observed in the wound sections (FIG. 13C). CD31 positive areas were enhanced in the CCL7 neutralizing antibody-treated mice, and the higher CD31 expressions in the wound area were detected in the CCL7 antibody-treated diabetic mice than in the untreated DM mice (FIG. 13D). In light of the above results of FIGS. 13A-13D, it was demonstrated that the inhibition of CCL7 by neutralizing antibody can improve wound repair in db/db type 2 diabetic mice.

Given the foregoing, the experiments demonstrate that the inhibition of CCL7 can improve the functions of HDMECs, so as to improve wound healing. The method of using a CCL7 antagonist of the present discourse is useful in improving the tube formation ability and/or the migration ability of endothelial cells, and thus is effective in improving would healing.

Even though numerous characteristics and advantages of The present disclosure have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

1. A method for improving would healing, comprising administering an effective amount of a chemokine C-C motif ligand 7 (CCL7) antagonist to a subject in need thereof to inhibit CCL7 activity.

2. The method according to claim 1, wherein the CCL7 antagonist is selected from a group consisting of a CCL7 neutralizing antibody, a CCL7 RNA interference (RNAi) agent, a C-C chemokine receptor type 1 antagonist, a C-C chemokine receptor type 2 antagonist, a C-C chemokine receptor type 3 antagonist, a C-C chemokine receptor type 5 antagonist, and a combination thereof.

3. The method according to claim 1, wherein the wound is a chronic wound.

4. The method according to claim 1, wherein the subject suffers from diabetic foot ulcer.

5. The method according to claim 1, wherein the effective amount of the CCL7 antagonist is from about 0.01 μg/kg to about 100 mg/kg.

6. The method according to claim 5, wherein the effective amount of the CCL7 antagonist is from about 0.1 μg/kg to about 1 mg/kg.

7. The method of claim 1, wherein the CCL7 antagonist is administered to the subject orally, sublingually, parenterally, rectally, intraperitoneally, intravenously, intradermally, intrapulmonarily, intramuscularly, subcutaneously, intrapleurally, topically, intranasally, or transdermally.

8. The method according to claim 1, wherein the administering enhances angiogenesis in the subject.

9. The method according to claim 1, wherein the administering protects endothelial cell functions in the subject.

10. The method according to claim 1, wherein a tube formation ability is improved.

11. The method according to claim 1, wherein a migration ability of endothelial cells is improved.

12. The method according to claim 10, wherein an expression of at least one inflammatory factor selected from the group consisting of G-CSF, IL-1β, IL-1α, IL-2, IL-6, IL-8, IL-11, IL-17, IL-18, IFN-α, IFN-β, IFN-γ, TNF-α, TNF-β, and a combination thereof is decreased.

13. The method according to claim 10, wherein an expression of an angiogenic factor is increased.

14. The method according to claim 13, wherein the angiogenic factor comprises VEGF, SDF-1, EGF, Ang, EPO, FGF, GDF, or a combination thereof.
